(19) United States
(12) Patent Application Publication
(10) Pub. No.: US 2022/0384020 A1
(43) Pub. Date: Dec. 1, 2022

(54) ESTIMATING METHOD, ESTIMATING DEVICE, AND ESTIMATING PROGRAM

(71) Applicant: NIPPON TELEGRAPH AND TELEPHONE CORPORATION, Tokyo (JP)

(72) Inventors: Atsuhiko MAEDA, Tokyo (JP); Kenichi FUKUDA, Tokyo (JP); Sun Yeong KIM, Tokyo (JP); Yukio KIKUYA, Tokyo (JP); Kazuaki OBANA, Tokyo (JP)

(73) Assignee: NIPPON TELEGRAPH AND TELEPHONE CORPORATION, Tokyo (JP)

(21) Appl. No.: 17/775,227

(22) PCT Filed: Nov. 8, 2019
(86) PCT No.: PCT/JP2019/043955

§ 371 Date: May 6, 2022

(51) Int. Cl. G16H 40/20

(52) U.S. Cl. CPC G16H 40/20

(57) ABSTRACT

A hospital that has a high possibility of accepting a user can be predicted with good precision. An estimating method is an estimating method of estimating a prospect of a hospital accepting a user at a first time. In the estimating method, a prospect of accepting the user at the first time is estimated by correlating a reason of the hospital rejecting acceptance of a user at a second time, and an amount of time from the second time to the first time.

### TECHNICAL FIELD

The technology of the disclosure relates to an estimating method, an estimating device, and an estimating program.

### BACKGROUND ART

When selecting a facility at which to be accepted in accordance with a user, there are cases in which searching for a facility that can accept the user is difficult. For example, a case is conceivable in which there is a request for emergency transportation from a patient, and a hospital to transport the patient to is searched for.

The number of ambulance calls is on the rise as of recent (see NPL 1). It is known that as one problem at the time of transporting a patent to a hospital by ambulance in response to a request for emergency transportation, identifying hospitals that are capable of accepting the patient is time consuming. In particular, when a transportation request is made to a hospital but is rejected thereby, and a hospital to transport the patient to has to be selected again, the time required for transportation may become markedly long.

In the flow of emergency transportation, generally, the patient is first carried into an ambulance, and thereafter a hospital to transport the patient to is searched for. Specifically, the ambulance crew members themselves search for a destination to transport the patient to, and accordingly may contact one hospital after another, or directly head to a hospital, in some cases. At this time, there are cases in which a hospital to transport the patient to is not found due to various factors such as physicians on duty, the state of examination and treatment being performed, and so forth, resulting in the patient being endlessly sent from one place to another (see NPL 2, for example).

### CITATION LIST

### Non Patent Literature

[NPL 1] “2017 White Paper on Fire Service”, Ministry of Internal Affairs and Communications, URL: https: //www.fdma.go.jp/publication/hakusho/h29/chapter2/sectio n5/45975.html
[NPL 2] “Byouin ‘taraimawashi’ no news ni hisomu shinkokusugiru genjitsu (A far too serious reality behind the news of hospital ‘taraimawashi’ (being sent from one hospital to another))”, URL: https://limo.media/articles/-/9406

### SUMMARY OF THE INVENTION

### Technical Problem

Several measures to solve such a phenomenon can be conceived. For example, conceivable measures include making a list of candidates for transportation destination hospitals, with symptoms of the user, outpatient history, and so forth, as a reference, and sending an email or the like to contact a plurality of hospitals at the same time to query whether capable of accepting or not.

However, simply making a list of hospitals necessitates contacting a plurality of hospitals regarding whether capable of accepting or not, consuming time until the transportation destination hospital is finalized. Also, in a case of contacting a plurality of hospitals at the same time to query whether capable of accepting or not, there are cases in which the ambulance and the hospitals need to be connected by network, and actions need to be actively undertaken at the hospital side, leaving unresolved issues.

The technology of the disclosure has been made with the foregoing in view, and it is an object thereof to provide an estimating method, an estimating device, and an estimating program that can predict, with good precision, a hospital of which the possibility of accepting a user is high.

### Means for Solving the Problem

A first aspect of the present disclosure is an estimating method of estimating a prospect of a hospital accepting a user at a first time. The estimating method causes a computer to execute processing including estimating the prospect of accepting the user at the first time, by correlating a reason of the hospital rejecting acceptance of a user at a second time further in the past from the first time, and an amount of time from the second time to the first time.

### Effects of the Invention

According to the technology of the disclosure, a hospital of which the possibility of accepting a user is high can be predicted with good precision.

### BRIEF DESCRIPTION OF DRAWINGS

FIG. 1 is a block diagram illustrating a configuration of a selection supporting device according to an embodiment.

FIG. 2 is a block diagram illustrating a hardware configuration of the selection supporting device.

FIG. 3 is a diagram showing an example of history data D1.

FIG. 4 is a diagram showing an example of prediction model generation data D2.

FIG. 5 is a diagram showing an example of prediction data D3.

FIG. 6 is a diagram showing an example of score calculation data D4.

FIG. 7 is a flowchart showing a flow of learning processing by the selection supporting device.

FIG. 8 is a flowchart showing a flow of prediction processing by the selection supporting device.

FIG. 9 is a diagram showing an example of vectors representing attribute information.

FIG. 10 is a diagram showing an example of processing of the prediction data D3.

FIG. 11 is a diagram showing an example of output data containing calculated acceptance probabilities.

### DESCRIPTION OF EMBODIMENTS

An example of an embodiment of technology according to the disclosure will be described below with reference to the Figures. Note that in the Figures, components and portions that are the same or equivalent are denoted by the same reference signs. Also, the dimensions and proportions in the Figures may be exaggerated for the sake of description, and differ from proportions in reality.

First, matters that were observed with regard to the technology according to the disclosure will be described. In the conventional technology described above, searching for a hospital is performed until a hospital that is capable of accepting is found. In other words, in a case in which acceptance is rejected, the processing ends once. However, it is assumed that depending on the reason for rejecting acceptance, there will be cases in which the reason is resolved by passage of time, and acceptance becomes possible. That is to say, a point was observed in that, by taking into consideration the reason for rejection, and the amount of time elapsed since the reply of the reason for rejection is received, precision of an estimation value of whether capable of accepting or not at the current point in time, and an estimation value of whether capable of accepting or not at a predetermined time, can be raised.

The configuration of the present embodiment will be described below. Note that an example of emergency transportation of a patient to a hospital, which is a facility, will be described below as an example. In a case of emergency transportation of a patient to a hospital, an ambulance crew member or an operator at a service center or the like, who is a user, selects a hospital for transportation to, and issues a transportation request (i.e., an acceptance request) to that hospital. However, this is not limited to cases of making requests to acceptance to a medical facility such as a hospital. For example, requests for acceptance of disaster victims to a shelter or the like in a disaster, requests for acceptance of materials to a facility that is a transportation destination of materials, and so forth, can be expected. Thus, the present disclosure provides technology enabling prediction of facilities of which the possibility of accepting requests from users is high, to support selection of facilities at which to be accepted.

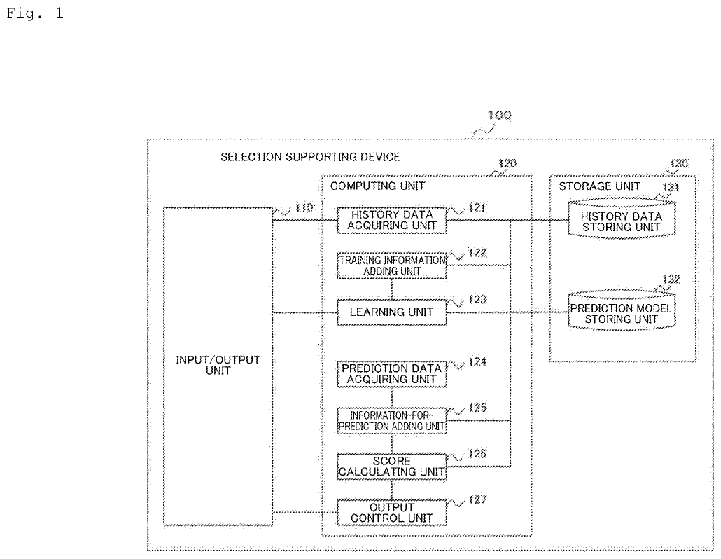

FIG. 1 is a block diagram illustrating the configuration of a selection supporting device according to the present embodiment. Note that the selection supporting device is an example of an estimating device according to the present disclosure.

As illustrated in FIG. 1, the selection supporting device 100 is provided with an input/output unit 110, a computing unit 120, and a storage unit 130.

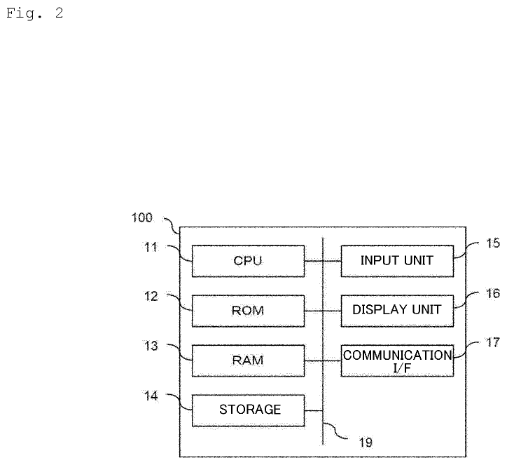

FIG. 2 is a block diagram illustrating the hardware configuration of the selection supporting device 100.

As illustrated in FIG. 2, the selection supporting device 100 has a CPU (Central Processing Unit) 11, ROM (Read Only Memory) 12, RAM (Random Access Memory) 13, storage 14, an input unit 15, display unit 16, and a communication interface (I/F) 17. The configurations are communicably connected to each other via a bus 19.

The CPU 11 is a central processing unit, and executes various types of programs, controls the parts, and so forth. That is to say, the CPU 11 reads out programs from the ROM 12 or the storage 14, and executes programs with the RAM 13 as a work region. The CPU 11 performs control of the above described configurations, and various types of computation processing, following programs stored in the ROM 12 or the storage 14. In the present embodiment, a selection support program is stored in the ROM 12 or the storage 14.

The ROM 12 stores various types of programs and various types of data. The RAM 13 temporarily stores programs and data as a work region. The storage 14 is configured of a storage device such as an HDD (Hard Disk Drive) or an SSD (Solid State Drive) or the like, and stores various types of programs including operating systems, and various types of data.

The input unit 15 includes a point device such as a mouse or the like, and a keyboard, and is used to perform various types of input.

The display unit 16 is, for example, a liquid crystal display, and displays various types of information. The display unit 16 may employ a touch panel system, and function as the input unit 15. Note that the input unit 15 and the display unit 16 correspond to the input/output unit 110.

The communication interface 17 is an interface for communicating with other equipment, such as terminal and so forth, and a standard such as, for example, Ethernet (a registered trademark), FDDI, Wi-Fi (a registered trademark), and so forth, is used.

The functional configurations of the selection supporting device 100 will be described next. The functional configurations are realized by the CPU 11 reading out the selection supporting program stored in the ROM 12 or the storage 14, loading to the RAM 13, and executing. Note that the selection supporting program is an example of an estimating program according to the present disclosure.

The computing unit 120 of the selection supporting device 100 includes a history data acquiring unit 121, a training information adding unit 122, a learning unit 123, a prediction data acquiring unit 124, an information-for-prediction adding unit 125, a score calculating unit 126, and an output control unit 127, as illustrated in FIG. 1. The storage unit 130 includes a history data storing unit 131 and a prediction model storing unit 132.

The history data acquiring unit 121 acquires history data relating to past acceptance requests from an input device, an external database, or the like, omitted from illustration, via the input/output unit 110, and stores in the history data storing unit 131. Acquisition of history data by the history data acquiring unit 121 is performed each time history data is accepted.

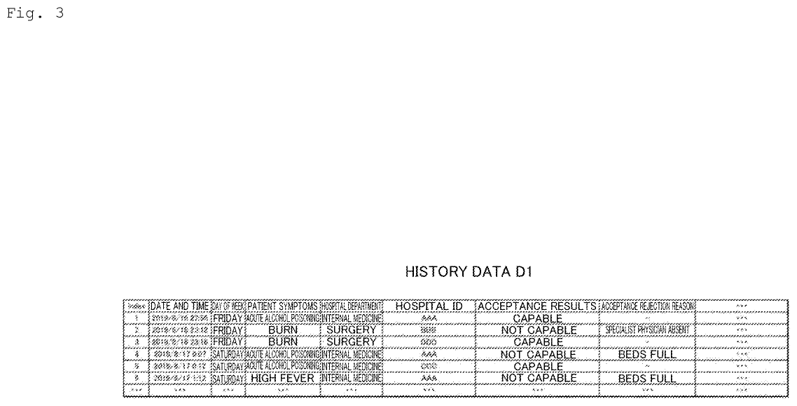

The history data storing unit 131 stores history data D1. FIG. 3 is a diagram showing an example of the history data D1. As shown in FIG. 3, the history data D1 is data in which attribute information, hospital ID, acceptance results, and acceptance rejection reason, for example, are correlated. The attribute information is various types of information relating to acceptance requests from a user. In a case of emergency transportation, the attribute information is, for example, the year, month, and date on which the request for emergency transportation was made, the day of the week thereof, the part of day thereof, symptoms of the patient, and the hospital department in accordance with the symptoms of the patient. The attribute information may also include the weather, facial complexion of the patient, heart rate, and so forth. Also, attribute information relating to candidate facilities may also be included. The hospital ID is identification information for the facility (hospital) to transport the patient to. The acceptance results are information of whether each facility accepted the acceptance request or not. The acceptance rejection reason is the rejection reason regarding when acceptance was rejected. It is sufficient for the acceptance rejection reason to be selected and input from a plurality of options prepared in advance, for example, which are the five of “in surgery”, “specialist physician absent”, “beds full”, “treatment difficult”, and “no response” here. Thus, the history data includes information relating to past acceptance requests, and information representing whether the acceptance requests were accepted or not.

The training information adding unit 122 reads out the history data D1 stored in the history data storing unit 131, generates prediction model generation data D2 used for generating a prediction model on the basis of this history data D1, and outputs to the learning unit 123. FIG. 4 is a diagram showing an example of the prediction model generation data D2. As illustrated in FIG. 4, the prediction model generation data D2 is data relating to the part of day, the day of the week, patient symptoms, hospital ID, acceptance results, most recent rejection reason in same hospital department, and time elapsed since rejection occurred. Generation of the prediction model generation data D2 will be described later.

The learning unit 123 executes processing of performing statistical analysis using vectors and correct labels obtained from the prediction model generation data D2, learns coefficient vectors as prediction models, and stores in the prediction model storing unit 132. The learning technique of prediction models will be described later. Note that settings may be made in advance so that the processing of the learning unit 123 is executed periodically (e.g., semimonthly, monthly, etc.).

The prediction model storing unit 132 stores a prediction model. The prediction model is used to predict, on the basis of attribute information relating to a newly-occurring acceptance request, the possibility of the candidate facilities accepting the acceptance request.

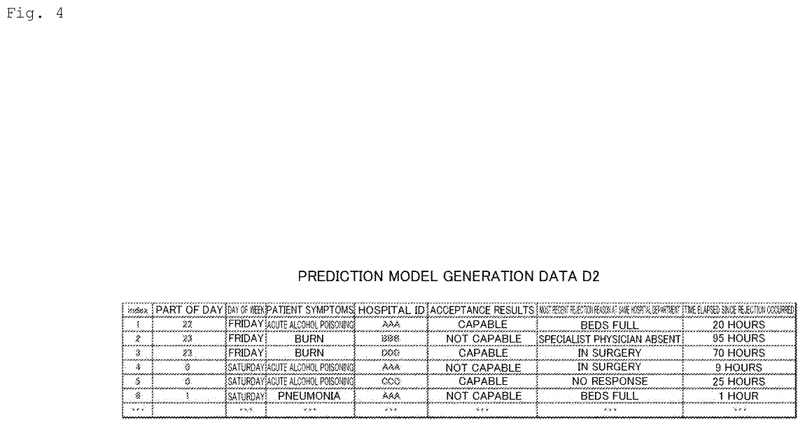

The prediction data acquiring unit 124 acquires prediction data D3 via the input/output unit 110, and outputs to the information-for-prediction adding unit 125. FIG. 5 is a diagram illustrating an example of the prediction data D3. As shown in FIG. 5, the prediction data D3 is data including, for example, the date and time of acceptance request, the day of the week, patient symptoms, and hospital department. This example shows that on Sep. 6, 2019, Friday, at 22:12, there was a patient exhibiting symptoms of acute alcohol poisoning, which is handled by the hospital department of internal medicine. In this way, the prediction data D3 is acquired regarding attribute information relating to acceptance requests including the date and time.

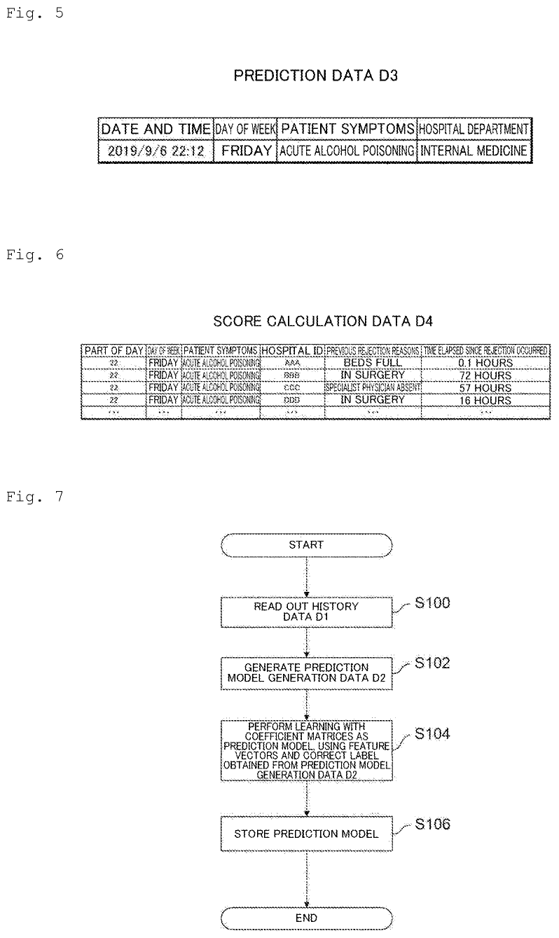

The information-for-prediction adding unit 125 generates score calculation data D4 on the basis of the acquired prediction data D3 and the history data D1 stored in the history data storing unit 131, and outputs to the score calculating unit 126. FIG. 6 is a diagram showing an example of the score calculation data D4. As shown in FIG. 6, the score calculation data D4 is data including, for example, the part of day, the day of the week, patient symptoms, hospital ID, previous rejection reasons, and time elapsed since rejection occurred. Generation of the score calculation data D4 will be described later.

The score calculating unit 126 calculates a score value for each hospital, on the basis of the score calculation data D4 output from the information-for-prediction adding unit 125 and the prediction model stored in the prediction model storing unit 132. The score value is a value representing the possibility of being accepted when an acceptance request is made to a particular facility. The calculation technique of the score value will be described later. Note that the score calculating unit 126 is an example of an estimating unit according to the present disclosure.

The output control unit 127 performs processing of creating output data on the basis of the score value calculated at the score calculating unit 126, and outputting via the input/output unit 110. The output processing will be described later.

Next, operations of the selection supporting device 100 will be described. The operations of the selection supporting device 100 are an example of the estimation method according to the present disclosure.

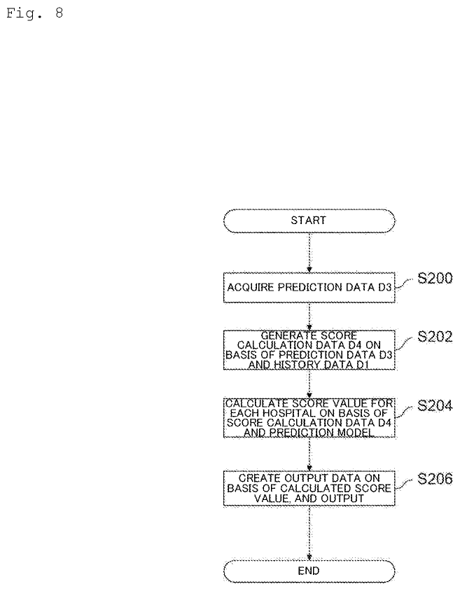

The selection supporting processing that is the operations of the selection supporting device 100 is divided into learning processing and prediction processing, and accordingly each processing will be described below. FIG. 7 is a flowchart showing the flow of learning processing by the selection supporting device 100. FIG. 8 is a flowchart showing the flow of prediction processing by the selection supporting device 100. Each processing is performed by the CPU 11 reading the selection supporting program out from the ROM 12 or the storage 14, and loading to the RAM 13 and executing.

First, the learning processing of the selection supporting device 100 will be described. In the present embodiment, the selection supporting device 100 learns a prediction model by the learning processing shown in FIG. 7. The prediction model is a model for predicting the likelihood of hospitals accepting transportation requests, i.e., the possibility of accepting acceptance requests. More specifically, in the present embodiment, generating of the prediction model refers to processing for calculating a coefficient matrix. A coefficient matrix is a matrix for calculating the score value representing the likelihood of hospitals accepting acceptance requests, and is a matrix learned as parameters with coefficients corresponding to vectors representing features. A later-described coefficient matrix W is an example of a first matrix, of which parameters that correspond to vectors representing attribute information according to the present disclosure are elements. A coefficient matrix V is an example of a second matrix of which parameters that correspond to vectors representing reasons of rejection are elements. Note that learning processing may be started at an optional timing, and for example may be automatically started every certain amount of time, or may be started triggered by an operation performed by an operator. Also, the history data D1 is acquired in advance by the history data acquiring unit 121, and is stored in the history data storing unit 131.

In step S100, the CPU 11 operates as the training information adding unit 122 and reads out the history data D1 stored in the history data storing unit 131.

In step S102, the CPU 11 operates as the training information adding unit 122 generates the prediction model generation data D2 used to generate the prediction model on the basis of the history data D1, and outputs to the learning unit 123.

Generation of the prediction model generation data D2 will be described. The training information adding unit 122 performs processing of calculating elapsed time for each row in the history data D1. In order to calculate the elapsed time, first, history data further in the past from the date and time of the object row is referenced, and rows in which acceptance rejection occurred at a date and time before the date and time of the object row are searched for from history data of the same hospital ID and the same hospital department. Next, for each row obtained from the search, the reason for the acceptance rejection thereof, and the calculation results of elapsed time from the date and time of the object row to the date and time of the rows obtained as the results of the search, are added to the object row. Now, the date and time before is a most recent date and time, for example. As an example, a case of adding the acceptance rejection and elapsed time to the sixth row of D1 shown in FIG. 3 will be described. In this case, with the sixth row as the object row, the acceptance rejection reason in the fourth row that is history data in the past from the date and time of the sixth row and that is of the same hospital ID and the same hospital department is extracted as the most recent rejection reason at the same hospital department, and the elapsed time is calculated. Also, the part of day information is calculated from the date and time information in D1, and unnecessary columns are deleted as necessary. The training information adding unit 122 outputs the prediction model generation data D2 generated in this way to the learning unit 123. Note that in the learning processing, the date and time of the object row is an example of a first time according to the present disclosure, and the date and time of the row obtained as a result of the search is an example of a second time that is in the past from the first time according to the present disclosure.

In step S104, the CPU 11 operates as the learning unit 123 and learns the coefficient matrix as a prediction model, by executing processing of statistical analysis using feature vectors and correct labels obtained from the prediction model generation data D2. Note that feature vectors will be written simply as vectors hereinafter.

Learning of the prediction model will be described. In the present embodiment, the learning unit 123 takes the acceptance results column (capable/not capable) in the prediction model generation data D2 as objective variables, and executes statistical analysis with all other information as explanatory variables (vectors). The acceptance results column is the correct label representing whether the hospitals are capable of accepting or not. Thus, coefficient matrices W and V for calculating score values that represent the likelihood of being accepted by hospitals, i.e., the level of the possibility of the acceptance request being accepted, are calculated as a prediction model. For example, in a case in which the acceptance results column shows capable, labeling of 1 is performed, and in a case of not capable, labeling of 0 is performed, and this is used as objective variables to perform statistical analysis.

For the statistical analysis executed by the learning unit 123, a technique such as, for example, logistic regression analysis, ranking learning, random forest, or the like, may be selected in accordance with the object. Here, a function f (p, h, Δt, r; W, V) is designed to output a great scalar value is as to a vector in a case where the acceptance request is “acceptable”. Note that here, p, h, Δt, and r are all included in the prediction model generation data D2 as vectors or variables. W and V represent coefficient matrices of which parameters that correspond to vectors are elements.

An example of a case of using logistic regression will be described below. First, the following Expression (1) is an expression to find a primary score value.

[Math. 1]

sᵢⱼ=pᵢ^{T}Whⱼ+exp(−Δtᵢⱼ)r_{i,j}^{T}Vhⱼ (1)

Here, pᵢ is a D×1-dimensional vector representing attribute information of the i'th patient (or patient acceptance request) occurring, and hⱼ is a vector representing, by one-hot encoding, a j count of hospitals that are acceptance candidates. r_{i,j} is a vector representing, by one-hot encoding, the reason for the acceptance rejection at the j′th hospital that has occurred at the most recent time, with the date and time of the acceptance request of the i′th patient occurring as a reference. Δt_{i,j} is a variable representing the elapsed time therebetween. The prediction model generation data D2 is not one-hot encoded, and accordingly encoding is performed as appropriate in cases of substituting into the above Expression (1). W and V are matrices of parameters that are to be found.

Now, the attribute information of the patient is made up of the 24 types of the part of day of issuing acceptance requests (every hour), the seven types of day of the week, and 50 types of cases, and with the number of hospitals that are acceptance candidates being 30, and reasons for acceptance rejection being five types, the details of the above Expression is as shown in Expression (2) below.

$$\begin{matrix}[ Math . 2 ] & \\ {s}_{i j} = {( \begin{matrix}{p}_{1 , i} \\ {p}_{2 , i} \\ ⋮ \\ {p}_{24 + 7 + 30 , i}\end{matrix} )}^{T} ( \begin{matrix}{w}_{1 , 1} & {w}_{1 , 2} & … & {w}_{1 , 30} \\ {w}_{2 , 1} & {w}_{2 , 2} & … & {w}_{2 , 30} \\ ⋮ & ⋮ & ⋱ & ⋮ \\ {w}_{24 + 7 + 30 , 1} & {w}_{24 + 7 + 30 , 2} & … & {w}_{24 + 7 + 50 , 30}\end{matrix} ) ( \begin{matrix}{h}_{1 , j} \\ {h}_{2 , j} \\ ⋮ \\ {h}_{30 , j}\end{matrix} ) + exp ( - Δ {t}_{i j} ) {( \begin{matrix}{r}_{1 , i , j} \\ {r}_{2 , i , j} \\ ⋮ \\ {r}_{8 , i , j}\end{matrix} )}^{T} ( \begin{matrix}{v}_{1 , 1} & {v}_{1 , 2} & … & {v}_{1 , 30} \\ {v}_{2 , 1} & {v}_{2 , 2} & … & {v}_{2 , 30} \\ ⋮ & ⋮ & ⋱ & ⋮ \\ {v}_{5 , 1} & {v}_{5 , 2} & … & {v}_{8 , 30}\end{matrix} ) ( \begin{matrix}{h}_{i , j} \\ {h}_{2 , j} \\ ⋮ \\ {h}_{30 , j}\end{matrix} ) & ( 2 )\end{matrix}$$

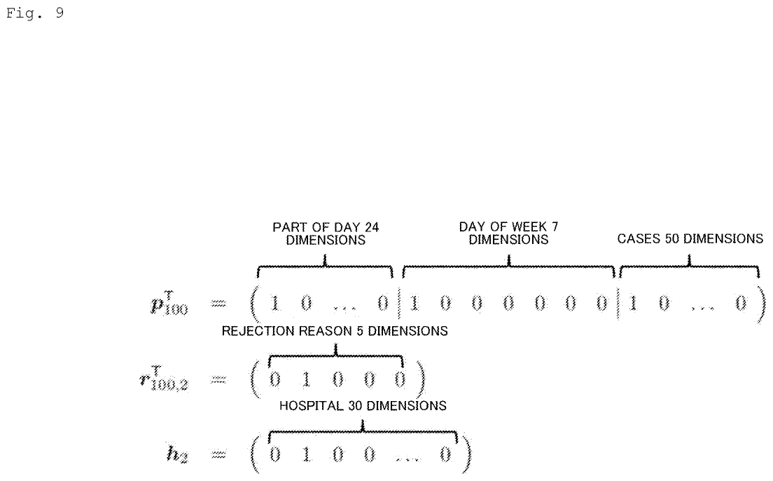

Vector pᵢ is a vector in which the part of day at which the i′th patient occurred, the day of the week of the occurrence, and patient case, are each one-hot encoded and combined. FIG. 9 is a diagram showing an example of vectors representing attribute information. The example in FIG. 9 shows an example of a vector p₁₀₀ representing attributes of a patient of index No. 100, an example of a vector r_{100,2} of the rejection reason occurring in a combination of the 100th patient and a 2nd hospital, and an example of a vector h₂ representing the 2nd hospital. Next, the coefficient matrices W and V will be described by way of example of parameters of the coefficient matrix W. A vector representing the part of day has parameters of w_{1,1}, w_{2,1}, . . . , w_{24,1}. In the same way, a vector representing the day of the week has parameters of w_{24+1,1}, w_{24+2,1}, . . . , w_{24+7,1}. The coefficient matrix W thus has parameters corresponding to vectors representing attribute information as elements thereof. In the same way, the coefficient matrix V has parameters corresponding to vectors representing the reason why hospitals rejected acceptance as elements thereof. An expression for logistic regression in which the above primary score value sᵢⱼ is substituted into the following logistic function and an acceptance probability pᵢⱼ for determining whether or not the patient will be accepted is found, is the following Expression (3).

$$\begin{matrix}[ Math . 3 ] & \\ {p}_{i j} = \frac{1}{1 + exp ( - {s}_{i j} )} & ( 3 )\end{matrix}$$

From the above expression, the probability of acceptance at each hospital regarding a patient with a particular case, occurring at a particular day of the week and a particular part of day, is found. The coefficient matrices W and V are found following the correct labels, so that the probability of acceptance is high when the correct label is capable of accepting: 1, and the probability of acceptance is low when the correct label is not capable of accepting: 0. Accordingly, the coefficient matrices W and V are learned as parameters such that take into consideration the reason for the most recent acceptance rejection, and the amount of time elapsed since the most recent occurrence of the reason of rejection, for each hospital.

In step S106, the CPU 11 operates as the learning unit 123 and stores the prediction model learned as the coefficient matrices W and V in the prediction model storing unit 132.

According to the above learning processing, parameters of the first matrix W and the second matrix V are learned as the prediction model according to the present disclosure, on the basis of a function that outputs values indicating the possibility of acceptance of a user for each hospital, and correct labels. The parameters for the first matrix W and the second matrix V are learned so that in the function, a value in which the possibility of acceptance is high is output, when the user is accepted. As described above, this function includes vector (pᵢ) representing predetermined attribute information relating to acceptance, and the first matrix (W) of which parameters corresponding to vectors representing the attribute information are elements. This function also includes the elapsed time (Δt_{i,j}) from the second time to the first time, vectors (r_{i,j}) representing reasons why hospitals rejected acceptance, and the second matrix (V) of which parameters corresponding to vectors representing reasons of rejection as elements.

This so far has been description of operations of the learning processing of the selection supporting device 100.

Next, operations of the prediction processing of the selection supporting device 100 will be described. In the present embodiment, the selection supporting device 100 performs prediction by the prediction processing shown in FIG. 8, and supports selection made by the user. This processing is started in accordance with input of a start request (acceptance request) from an ambulance crew member or an operator at a service center for example, when there is an occurrence in which a new patient needs emergency transportation, for example.

In step S200, the CPU 11 operates as the prediction data acquiring unit 124 and acquires the prediction data D3 via the input/output unit 110, and outputs to the information-for-prediction adding unit 125.

The prediction data D3 includes attribute information relating to emergency transportation newly requested from a patient, as an acceptance request that has newly occurred. More specifically, attribute information includes, in addition to environmental information such as the date and time, the day of the week, and so forth, hospital department in accordance with symptoms of the planned transportee (patient), and patient information such as symptoms. An example of the prediction data D3 is as illustrated in FIG. 5.

In step S202, the CPU 11 operates as the information-for-prediction adding unit 125 and generates the score calculation data D4 on the basis of the acquired prediction data D3 and the history data D1 stored in the history data storing unit 131, and outputs to the score calculating unit 126.

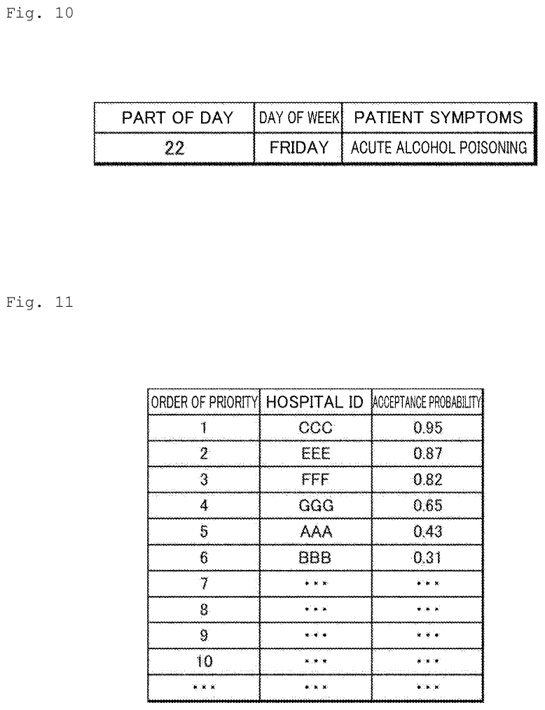

The process of generating the score calculation data D4 will be described. First, the date and time in the prediction data D3 is converted into part of day information as in FIG. 10, and unnecessary information is processed. Next, the CPU 11 references the history data D1 stored in the history data storing unit 131, and searches for, with regard to each hospital, a row in which the hospital department is the same as that of the prediction data D3, and also in which an acceptance rejection occurred at a date and time before than the current time (the date and time in the prediction data D3). The reason for the acceptance rejection is extracted from the row found as a result of the search, and the elapsed time from the date and time of the row obtained as this search result to the current date and time is calculated. The date and time before than the current time is the most recent date and time, for example. As for a method of selecting a date and time other than the most recent, if a further acceptance rejection has occurred immediately prior thereto, and acceptance rejections are continuously occurring immediately therebefore for the same reason, the row with oldest time out of the continuous occurrences may be taken as the object. The reason thereof is to aim to raise prediction precision by standardizing data so as to reference as early a time as possible at which this situation in which acceptance is difficult occurred. The CPU 11 then creates duplicates of the data in FIG. 10 for each of the hospitals, combines the data of the hospitals, thereby creating the score calculation data D4, and outputs the score calculation data D4 to the score calculating unit 126. An example of the score calculation data D4 is as shown in FIG. 6. Thus, the score calculation data D4 includes attribute information for each hospital, elapsed time, and reason for acceptance rejection by the hospital. Note that in the prediction processing, the date and time in the prediction data D3 is an example of the first time according to the present disclosure, and the date and time of the row obtained from the history data D1 as the search result is an example of the second time according to the present disclosure. Also, as described above, the second time is a time at which acceptance by a hospital has been rejected most recently, with the first time as a reference. Also, in a case of a plurality of times of acceptance rejection occurring within a predetermined amount of time with the second time as a reference, the time at which acceptance rejection occurred the farthest in the past within the predetermined amount of time may be set as a new second time.

In step S204, the CPU 11 operates as the score calculating unit 126, and calculates a score value for each hospital, on the basis of the score calculation data D4 output from the information-for-prediction adding unit 125 and the prediction model stored in the prediction model storing unit 132. The score value for each hospital is an example of a value indicating the possibility of acceptance for each hospital according to the present disclosure.

An example of calculating a score value will be described. First, the score calculating unit 126 acquires the coefficient matrices W and V as the prediction model stored in the prediction model storing unit 132. The attribute information, the hospital ID, and the acceptance rejection reason, in each row in the score calculation data D4, is vectorized here in accordance with the format in FIG. 9. The score value for each hospital is obtained by calculating the acceptance probability pᵢⱼ for each hospital, by applying this vector and elapsed time to the above Expression (2) and Expression (3) using the coefficient matrices W and V as the prediction model, for each hospital j. The acceptance probability represents the likelihood of requests being accepted with regard to each hospital, and the higher the acceptance probability is, this means that the more likely the acceptance request is to be accepted. As described above, a score value is found for each hospital j regarding the patient acceptance request i.

In step S206, the CPU 11 operates as the output control unit 127 and performs processing of creating output data on the basis of the calculate score values, and outputting via the input/output unit 110.

Output data will be described. For example, the output control unit 127 can create a priority list, in which a plurality of hospitals that are candidates for transporting have been prioritized by being sorted in descending order of the calculated acceptance probabilities, as output data. The calculated acceptance probabilities may be used as output data without change here, or may be used as output data in which those other than the highest ranking in the sorting are deleted. Moreover, in a case where the distance from the location where the patient occurred to each hospital is known in advance, the hospitals to be displayed may be narrowed down by providing a threshold value for the distance, or alternatively, the distance and the acceptance probability may be displayed as a set.

According to the above prediction processing, the estimation method according to the present disclosure is executed as an estimation method for estimating the prospect of a hospital accepting a user at the first time. Thus, the prospect of the user being accepted at the first time is estimated by correlating the reason of the hospital rejecting acceptance of the user at the second time, and the time from the second time to the first time. Note that acceptance of the user is acceptance of some sort of action taken by the user with regard to a facility, and indicates accepting of an acceptance request issued from the user to a hospital, or the like, for example.

FIG. 11 is a diagram illustrating an example of output data including the calculated acceptance probability. FIG. 11 shows a priority list, sorted in descending order from hospitals with a high probability to hospitals with a low probability, on the basis of the calculated acceptance probability as output data. This indicates that the higher the acceptance probability is for the hospitals, the higher the possibility is that a transportation request will be accepted is. Accordingly, in the priority list in FIG. 11, hospital CCC that has the highest score value of 0.95 has the greatest possibility of accepting a transportation request. Also, hospital EEE that has the second highest possibility of accepting a transportation request has a score value of 0.87, and the third is hospital FFF exhibiting a score value of 0.82. By outputting this priority list as output data, an ambulance crew member or an operator, who is a user, can immediately judge that the hospital CCC has the highest possibility of accepting the current transportation request, by viewing the priority list, and issue to transportation request to the hospital CCC. Even in a case where acceptance by the hospital CCC is rejected, the second hospital EEE that is the next candidate can be promptly selected, and accordingly the amount of time necessary of selecting candidates for transportation to can be minimized. Also, facility names may be output instead of hospital IDs, to improve user convenience.

Also, in a case of issuing a transportation request to one of the hospitals in the above, and the results are found, the ambulance crew member or operator, who is a user, immediately transmits the results to the selection supporting device 100. The history data in the history data storing unit 131 is immediately updated by the history data acquiring unit 121 in light of the results. Thus, the history data of the history data storing unit 131 is constantly maintained in a newest state, and prediction precision of a location at which to be accepted can be raised by using the newest information of acceptance rejection at each hospital.

As described above, according to the selection supporting device 100 of the present embodiment, hospitals with a high possibility of accepting a user can be predicted with good precision.

Although an example has been described in the above embodiment regarding a case of predicting the prospect of an acceptance request of a user being accepted at a facility that is a hospital, this example is not limiting. For example, application may also be made to a case of replacing the facilities with Web advertisements, setting individual features as some sort of attributes of the Web advertisement side, and finding the probability of a user having a certain attribute clicking on each Web advertisement. In this case, the Web advertisements are an example of a predetermined object.

Note that various types of processors other than a CPU may execute the learning processing or prediction processing that the CPU executes by reading in software (programs) and executing in the above embodiments. Examples of processors in this case include dedicated electric circuits and so forth that are processors having a circuit configuration designed for dedicated execution of particular processing, such as PLDs (Programmable Logic Device) of which the circuit configuration can be changed after manufacturing, like FPGAs (Field-Programmable Gate Array) and so forth, and ASICs (Application Specific Integrated Circuit) and so forth. This learning processing or prediction processing may also be executed by one of these various types of processors, or may be executed by a combination of two or more processors of the same type or different types (e.g., a plurality of FPGAs, a combination of a CPU and an FPGA, and so forth). More specifically, the hardware configuration of these various types of processors are electric circuits where circuit elements such as semiconductor elements and so forth are combined.

Also, while description is made in the above embodiments regarding an arrangement in which a selection supporting program is stored (installed) in the storage 14 in advance, this is not limiting. The program may be provided in a form stored in a non-transitory storage medium, such as a CD-ROM (Compact Disk Read Only Memory), DVD-ROM (Digital Versatile Disk Read Only Memory), USB (Universal Serial Bus) memory, and so forth. A form may also be made in which the program is downloaded from an external device via a network.

In relation to the above embodiment, the following appendices are further disclosed.

### Appendix 1

An estimating device, including:

a memory, and

at least one processor connected to the memory,

wherein the processor

is an estimating device that estimates a prospect of a hospital accepting a user at a first time, and

is configured to estimate the prospect of acceptance of the user at the first time, by correlating a reason of the hospital rejecting acceptance of a user at a second time further in the past from the first time, and an amount of time from the second time to the first time.

### Appendix 2

A non-transitory storage medium storing an estimating program

that is an estimating program that estimates a prospect of a hospital accepting a user at a first time, and

that causes a computer to execute

estimating of the prospect of accepting the user at the first time, by correlating a reason of the hospital rejecting acceptance of a user at a second time further in the past from the first time, and an amount of time from the second time to the first time.

### REFERENCE SIGNS LIST

100 Selection supporting device
110 Input/output unit
120 Computing unit
121 History data acquiring unit
122 Training information adding unit
123 Learning unit
124 Prediction data acquiring unit
125 Information-for-prediction adding unit
126 Score calculating unit
127 Output control unit
130 Storage unit
131 History data storing unit
132 Prediction model storing unit

1. An estimating method of estimating a prospect of a hospital accepting a user at a first time, the estimating method causing a computer to execute processing comprising:
estimating a prospect of accepting the user at the first time, by correlating a reason of the hospital rejecting acceptance of a user at a second time further in the past from the first time, and an amount of time from the second time to the first time.

2. The estimating method according to claim 1, wherein the second time is a most recent time at which acceptance at the hospital was rejected, with the first time as a reference.

3. The estimating method according to claim 2, wherein, when a plurality of times of acceptance rejection occurs within a predetermined amount of time with the second time as a reference, the second time includes a time at which acceptance rejection occurred the farthest in the past within the predetermined amount of time.

4. The estimating method according to claim 1, wherein the correlating further includes predicting a value indicating a possibility of acceptance of the user for each hospital, using a prediction model that has performed learning in advance,
wherein the prediction model has learned parameters of a first matrix and a second matrix so as to output a value in which the possibility of acceptance is high in a function in a case in which the user is accepted, on the basis of the function that outputs a value indicating a possibility of acceptance of the user, and that includes a vector representing predetermined attribute information relating to acceptance, the first matrix having a parameter corresponding to the vector representing the attribute information as an element, elapsed time from the second time to the first time, a vector representing a reason of the hospital rejecting acceptance, and the second matrix having a parameter corresponding to the vector representing the reason of the rejection as an element, and on a correct label representing whether the hospital is capable of accepting or not.

5. An estimating method causing a computer to execute processing including:
with a first time as a current time at which an acceptance request occurred, and a second time as a time at which an acceptance rejection occurred at a date and time before the current time,
generating data for score calculation, on the basis of data for prediction that is acquired regarding attribute information relating to an acceptance request including a date and time, and history data in which are correlated attribute information, hospital identification information, an acceptance result, and an acceptance rejection reason, the data for score calculation including, for each hospital, attribute information of the acceptance request, elapsed time from the second time to the first time, and the acceptance rejection reason at the hospital at the second time; and
calculating a score value for each hospital, on the basis of the data for score calculation that is generated, and a prediction model that has performed learning in advance to output a score value indicating the possibility of acceptance at each hospital, by calculating an acceptance probability by applying the prediction model, with the attribute information, the elapsed time, and the acceptance rejection reason, in the data for score calculation, as vectors.

6. An estimating method of estimating a prospect of a predetermined object accepting a user at a first time, the estimating method causing a computer to execute processing comprising:
estimating a prospect of accepting the user at the first time, by correlating a reason of the object rejecting acceptance of a user at a second time further in the past from the first time, and an amount of time from the second time to the first time.

7-8. (canceled)

9. The estimating method according to claim 1, wherein the reason of the hospital rejecting the user includes one of: in surgery, specialist physician absent, beds full, treatment difficult, or no response.

10. The estimating method according to claim 2, wherein the correlating further includes predicting a value indicating a possibility of acceptance of the user for each hospital, using a prediction model that has performed learning in advance,
wherein the prediction model has learned parameters of a first matrix and a second matrix so as to output a value in which the possibility of acceptance is high in a function in a case in which the user is accepted, on the basis of the function that outputs a value indicating a possibility of acceptance of the user, and that includes a vector representing predetermined attribute information relating to acceptance, the first matrix having a parameter corresponding to the vector representing the attribute information as an element, elapsed time from the second time to the first time, a vector representing a reason of the hospital rejecting acceptance, and the second matrix having a parameter corresponding to the vector representing the reason of the rejection as an element, and on a correct label representing whether the hospital is capable of accepting or not.

11. The estimating method according to claim 3, wherein the correlating further includes predicting a value indicating a possibility of acceptance of the user for each hospital, using a prediction model that has performed learning in advance,
wherein the prediction model has learned parameters of a first matrix and a second matrix so as to output a value in which the possibility of acceptance is high in a function in a case in which the user is accepted, on the basis of the function that outputs a value indicating a possibility of acceptance of the user, and that includes a vector representing predetermined attribute information relating to acceptance, the first matrix having a parameter corresponding to the vector representing the attribute information as an element, elapsed time from the second time to the first time, a vector representing a reason of the hospital rejecting acceptance, and the second matrix having a parameter corresponding to the vector representing the reason of the rejection as an element, and on a correct label representing whether the hospital is capable of accepting or not.

12. The estimating method according to claim 5, wherein the second time is a most recent time at which acceptance at the hospital was rejected, with the first time as a reference.

13. The estimating method according to claim 12, wherein, when a plurality of times of acceptance rejection occurs within a predetermined amount of time with the second time as a reference, the second time includes a time at which acceptance rejection occurred the farthest in the past within the predetermined amount of time.

14. The estimating method according to claim 12, wherein the correlating further includes predicting a value indicating a possibility of acceptance of the user for each hospital, using a prediction model that has performed learning in advance,
wherein the prediction model has learned parameters of a first matrix and a second matrix so as to output a value in which the possibility of acceptance is high in a function in a case in which the user is accepted, on the basis of the function that outputs a value indicating a possibility of acceptance of the user, and that includes a vector representing predetermined attribute information relating to acceptance, the first matrix having a parameter corresponding to the vector representing the attribute information as an element, elapsed time from the second time to the first time, a vector representing a reason of the hospital rejecting acceptance, and the second matrix having a parameter corresponding to the vector representing the reason of the rejection as an element, and on a correct label representing whether the hospital is capable of accepting or not.

15. The estimating method according to claim 12, wherein the reason of the hospital rejecting the user includes one of: in surgery, specialist physician absent, beds full, treatment difficult, or no response.

16. The estimating method according to claim 6, wherein the second time is a most recent time at which acceptance at the hospital was rejected, with the first time as a reference.

17. The estimating method according to claim 16, wherein, when a plurality of times of acceptance rejection occurs within a predetermined amount of time with the second time as a reference, the second time includes a time at which acceptance rejection occurred the farthest in the past within the predetermined amount of time.

18. The estimating method according to claim 16, wherein the reason of the hospital rejecting the user includes one of: in surgery, specialist physician absent, beds full, treatment difficult, or no response.

19. The estimating method according to claim 16, wherein the correlating further includes predicting a value indicating a possibility of acceptance of the user for each hospital, using a prediction model that has performed learning in advance,
wherein the prediction model has learned parameters of a first matrix and a second matrix so as to output a value in which the possibility of acceptance is high in a function in a case in which the user is accepted, on the basis of the function that outputs a value indicating a possibility of acceptance of the user, and that includes a vector representing predetermined attribute information relating to acceptance, the first matrix having a parameter corresponding to the vector representing the attribute information as an element, elapsed time from the second time to the first time, a vector representing a reason of the hospital rejecting acceptance, and the second matrix having a parameter corresponding to the vector representing the reason of the rejection as an element, and on a correct label representing whether the hospital is capable of accepting or not.
